(19) United States
(12) Patent Application Publication
(10) Pub. No.: US 2009/0292179 A1
(43) Pub. Date: Nov. 26, 2009

(54) MEDICAL SYSTEM HAVING A MEDICAL UNIT AND A DISPLAY MONITOR

(75) Inventors: Hemant Jampala, Chennai (IN); Ross G. Krogh, Cincinnati, OH (US); David Q. Feng, Mason, OH (US); Donn C. Mueller, Mason, OH (US); Paul J. Niklewski, Cincinnati, OH (US)

Correspondence Address: THOMPSON HINE L.L.P.;Intellectual Property Group, P.O. BOX 8801, DAYTON, OH, 45401-8801, US

(73) Assignee: ETHICON ENDO-SURGERY, INC., Cincinnati, OH (US)

(21) Appl. No.: 12/124,649

(22) Filed: May 21, 2008

(51) Int. Cl. A61B 5/00

(52) U.S. Cl. 600/301

(57) ABSTRACT

A medical system includes a display monitor and a medical unit. The medical unit is adapted to measure a physiological parameter of a patient. The medical unit is adapted to wirelessly connect to the display monitor, wherein the display monitor is adapted to display the physiological parameter. In one implementation of the medical system, the medical unit is adapted to automatically wirelessly connect to the display monitor when the medical unit is brought into proximity with the display monitor. In one illustration of the medical system, the physiological parameter is a pulse rate of the patient.

### FIELD OF THE INVENTION

The present invention is related generally to medical technology, and more particularly to a medical system having a medical unit and a display monitor, wherein the medical unit is adapted to measure a physiological parameter of a patient.

### BACKGROUND OF THE INVENTION

Known medical systems include those having a medical unit which measures a physiological parameter of a patient. One known example of such a medical system is a drugstore digital thermometer which is a medical unit having a temperature-measuring module and having a built-in LCD (liquid crystal display) display monitor, wherein the temperature-measuring module is connected to the display monitor by an electrical conductor, and wherein the display monitor displays a temperature reading calculated by the temperature-measuring module.

Another known example of such a medical system is a preconfigured conscious sedation system having a bedside monitoring unit including a pulse oximeter which measures the pulse rate and the SpO₂ (saturation of peripheral oxygen) level of the patient and including a blood pressure module which measures the systolic blood pressure level and the diastolic blood pressure level of the patient. The bedside monitoring unit travels with the patient from the preparation room to the procedure room. Then, the bedside monitoring unit is connected by a cable to a procedure room unit which has a wired display monitor, wherein the pulse rate, the SpO₂ respiration rate, the systolic blood pressure level, and the diastolic blood pressure level (and other preconfigured physiological parameters of the patient) measured by the bedside monitoring unit are displayed on the display monitor. The procedure room unit delivers a sedation drug intravenously to the patient at a flow rate, wherein the display monitor displays the flow rate of the delivered sedation drug.

Still, scientists and engineers continue to seek improved medical systems having a medical unit and a display monitor, wherein the medical unit is adapted to measure a physiological parameter of a patient.

### SUMMARY

A first expression of an embodiment of the invention is for a medical system including a display monitor and a first medical unit. The first medical unit is adapted to measure a first physiological parameter of a patient. The first medical unit is adapted to wirelessly connect to the display monitor, wherein the display monitor is adapted to display the first physiological parameter.

A second expression of an embodiment of the invention is for a medical system including a display monitor, a first medical unit, and a second medical unit. The first medical unit is adapted to measure a first physiological parameter of a patient and is adapted to wirelessly connect to the display monitor separately from the wireless connection of the first medical unit to the display monitor, wherein the display monitor is adapted to display the first physiological parameter. The second medical unit is adapted to measure a second physiological parameter of the patient different from the first physiological parameter and is adapted to wirelessly connect to the display monitor, wherein the display monitor is adapted to display the second physiological parameter.

A third expression of an embodiment of the invention is for a medical system including a display monitor and a first medical unit. The first medical unit is adapted to measure a first physiological parameter of a patient and to deliver a first drug to the patient. The first medical unit is adapted to wirelessly connect to the display monitor, wherein the display monitor is adapted to display the first physiological parameter and a flow rate of the first drug.

A fourth expression of an embodiment of the invention is for a medical system including a display monitor, a first medical unit, and a second medical unit. The first medical unit is adapted to measure a first physiological parameter of a patient and is adapted to wirelessly connect to the display monitor, wherein the display monitor is adapted to display the first physiological parameter. The second medical unit is adapted to deliver a second drug to the patient and is adapted to wirelessly connect to the display monitor separately from the wireless connection of the first medical unit to the display monitor, wherein the display monitor is adapted to display the flow rate of the second drug.

Several benefits and advantages are obtained from one or more of the expressions of the embodiment of the invention. In one example, the wireless connection reduces the number of cables present during a medical procedure. In the same or a different example, each medical unit is adapted to automatically wirelessly connect to the display monitor when brought into proximity with the display monitor which reduces the time the procedure room is occupied to perform a medical procedure on a patient.

### BRIEF DESCRIPTION OF THE FIGURES

FIG. 1 is schematic diagram of an embodiment of a medical system of the invention wherein two medical units are operatively connected to a patient and wherein dashed arrowhead lines represent wireless connections of the two medical units to a display monitor; and

FIG. 2 is a schematic diagram of the display monitor of the medical system of FIG. 1, wherein the display monitor is displaying medical information.

### DETAILED DESCRIPTION

Before explaining the expressions of an embodiment of the invention in detail, it should be noted that each is not limited in its application or use to the details of construction and arrangement of parts, instructions, and steps illustrated in the accompanying drawings and description. The illustrative embodiment of the invention may be implemented or incorporated in other embodiments, variations, and modifications, and may be practiced or carried out in various ways. Furthermore, unless otherwise indicated, the terminology employed herein has been chosen for the purpose of describing the illustrative expressions of the embodiment of the present invention for the convenience of the reader and not for the purpose of limiting the invention.

It is further understood that any one or more of the following-described expressions of a medical system, implementations, etc. can be combined with any one or more of the other following-described expressions of a medical system, implementations, etc.

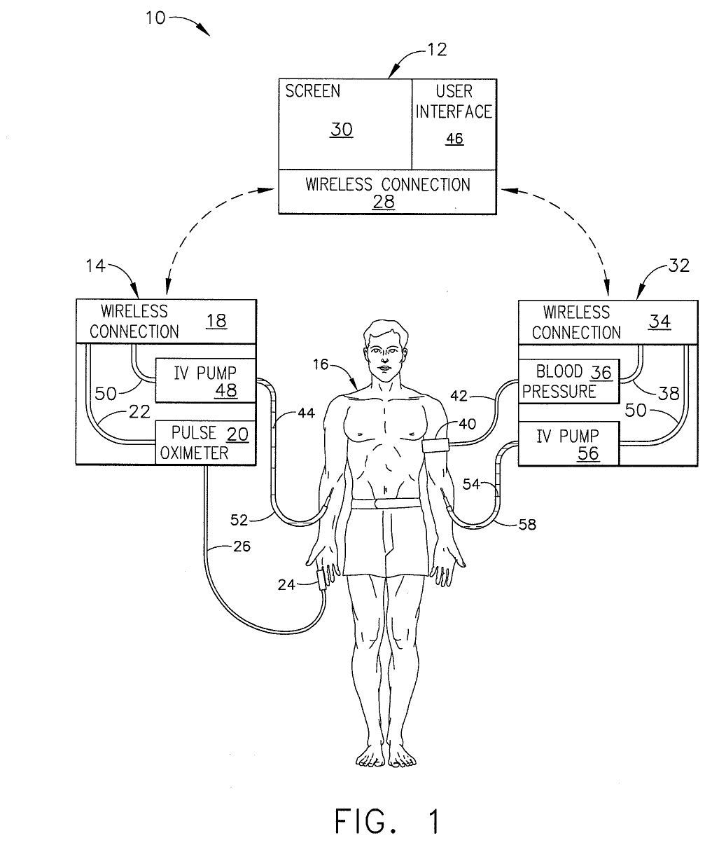

An embodiment of the invention is shown in FIG. 1. A first expression of the embodiment of FIG. 1 is for a medical system 10 including a display monitor 12 and a first medical unit 14. The first medical unit 14 is adapted to measure a first physiological parameter of a patient 16. The first medical unit 14 is adapted to wirelessly connect to the display monitor 12, wherein the display monitor 12 is adapted to display the first physiological parameter.

Examples of patient physiological parameters include, without limitation, ECG (electrocardiogram) data, blood pressure data, pulse rate data, SpO₂ (saturation of peripheral oxygen) data, etc. Examples of wireless connection (i.e., a connection without an electrical conductor) modules suitable for use in an instrumented medical environment include, without limitation, Bluetooth® technology, IR (infrared), ultrasound, long range RFID (radio frequency identification), etc.

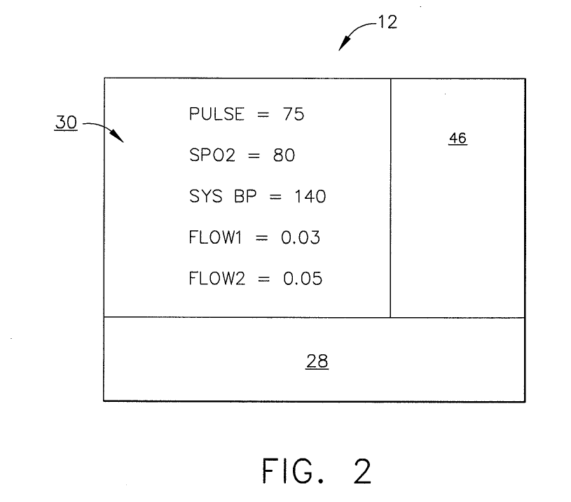

In one implementation of the first expression of the embodiment of FIGS. 1-2, the first medical unit 14 is adapted to automatically wirelessly connect to the display monitor 12 when the first medical unit 14 is brought into proximity with the display monitor 12. In one example, Bluetooth® technology is used for the automatic wireless connection. In a different implementation, not shown, a user interface on the first medical unit and/or a user interface on the display monitor enables a user to make the wireless connection.

In one enablement of the first expression of the embodiment of FIGS. 1-2, the first medical unit 14 is adapted to measure an additional physiological parameter of the patient 16 different from the first physiological parameter, and the display monitor 12 is adapted to display the additional physiological parameter. In one illustration, the first physiological parameter is a pulse rate of the patient 16, and the additional physiological parameter is an SpO₂ level of the patient 16.

In one construction of the first expression of the embodiment of FIGS. 1-2, the first medical unit 14 includes a wireless connection module 18 which is connected to a pulse oximeter module 20 by a conductor 22, wherein the pulse oximeter module 20 is connected to a pulse oximeter sensor 24 by a cable 26. The pulse oximeter sensor 24 is disposable on the patient 16, the wireless connection module 18 is adapted to wirelessly connect to a corresponding wireless connection module 28 in the display monitor 12, and the display monitor 12 is adapted to display the first physiological parameter on a screen 30 of the display monitor 12. In one variation, the wireless connection module 18 of the first medical unit 14 sends a wireless signal or segment thereof which includes an identification portion which is recognized by the wireless connection module 28 of the display monitor 12 as a signal from a pulse oximeter module and which includes a pulse rate data portion and an SpO₂ level data portion recognizable as such by the wireless connection module 28 of the display monitor 12.

A second expression of the embodiment of FIGS. 1-2 is for a medical system 10 including a display monitor 12, a first medical unit 14, and a second medical unit 32. The first medical unit 14 is adapted to measure a first physiological parameter of a patient 16 and is adapted to wirelessly connect to the display monitor 12, wherein the display monitor 12 is adapted to display the first physiological parameter. The second medical unit 32 is adapted to measure a second physiological parameter of the patient 16 different from the first physiological parameter and is adapted to wirelessly connect to the display monitor 12 separately from the wireless connection of the first medical unit 14 to the display monitor 12, wherein the display monitor 12 is adapted to display the second physiological parameter.

In one enablement of the second expression of the embodiment of FIGS. 1-2, the first physiological parameter is a pulse rate of the patient 16, and the second physiological parameter is a systolic blood pressure level of the patient 16. In one implementation, the first medical unit 14 is adapted to automatically wirelessly connect to the display monitor 12 when the first medical unit 14 is brought into proximity with the display monitor 12. In the same or a different implementation, the second medical unit 32 is adapted to automatically wirelessly connect to the display monitor 12 when the second medical unit 32 is brought into proximity with the display monitor 12.

In one construction of the second expression of the embodiment of FIGS. 1-2, the second medical unit 32 includes a wireless connection module 34 which is connected to a blood pressure module 36 by a conductor 38, wherein the blood pressure module 36 is connected to a blood pressure cuff 40 by a tube 42. The blood pressure cuff 40 is disposable on the patient 16, the wireless connection module 34 is adapted to wirelessly connect to the corresponding wireless connection module 28 in the display monitor 12, and the display monitor 12 is adapted to display the second physiological parameter on the screen 30 of the display monitor 12. In one variation, the wireless connection module 34 of the second medical unit 32 sends a wireless signal or segment thereof which includes an identification portion which is recognized by the wireless connection module 28 of the display monitor 12 as a signal from a blood pressure module 36 and which includes a systolic blood pressure level data portion and a diastolic blood pressure level data portion recognizable as such by the wireless connection module 28 of the display monitor 12.

A third expression of the embodiment of FIGS. 1-2 is for a medical system 10 including a display monitor 12 and a first medical unit 14. The first medical unit 14 is adapted to measure a first physiological parameter of a patient 16 and to deliver a first drug 44 to the patient 16. The first medical unit 14 is adapted to wirelessly connect to the display monitor 12, wherein the display monitor 12 is adapted to display the first physiological parameter and a flow rate of the first drug 44.

In one implementation of the third expression of the embodiment of FIGS. 1-2, the wireless connection of the first medical unit 14 to the display monitor 12 is a two-way connection, and the display monitor 12 includes a user interface 46 for changing the flow rate of the first drug 44. In one illustration, the first drug 44 is a sedation drug (such as Propofol used as a conscious sedation drug). In the same or a different illustration, the first physiological parameter is a pulse rate of the patient 16. In one implementation, the first medical unit 14 is adapted to automatically wirelessly connect to the display monitor 12 when the first medical unit 14 is brought into proximity with the display monitor 12.

In one construction of the third expression of the embodiment of FIGS. 1-2, the first medical unit 14 includes a first IV (intravenous) pump assembly 48 which is connected to the wireless connection module 18 of the first medical unit 14 by a wire 50, which is controllable from the user interface 46 of the display monitor 12, and which propels the first drug 44 through a first IV tube 52 to the patient 16. In one variation, the wireless connection module 18 of the first medical unit 14 sends a wireless signal or segment thereof which includes an identification portion which is recognized by the wireless connection module 28 of the display monitor 12 as a signal from a first IV pump assembly 48 and which includes a flow rate data portion recognizable as such by the wireless connection module 28 of the display monitor 12. In a different construction, not shown, inhalation drug delivery using pressurized gas is employed by way of an oral and/or nasal cannula. Other constructions are left to the artisan.

A fourth expression of the embodiment of FIGS. 1-2 is for a medical system 10 including a display monitor 12, a first medical unit 14, and a second medical unit 32. The first medical unit 14 is adapted to measure a first physiological parameter of a patient 16 and is adapted to wirelessly connect to the display monitor 12, wherein the display monitor 12 is adapted to display the first physiological parameter. The second medical unit 32 is adapted to deliver a second drug 54 to the patient 16 and is adapted to wirelessly connect to the display monitor 12 separately from the wireless connection of the first medical unit 14 to the display monitor 12, wherein the display monitor 12 is adapted to display the flow rate of the second drug 54.

In one implementation of the fourth expression of the embodiment of FIGS. 1-2, the wireless connection of the second medical unit 32 to the display monitor 12 is a two-way connection, and the display monitor 12 includes a user interface 46 for changing the flow rate of the second drug 54. In one illustration, the second drug 54 is a sedation drug. In the same or a different illustration, the first physiological parameter is a pulse rate of the patient 16. In one implementation, the first medical unit 14 is adapted to automatically wirelessly connect to the display monitor 12 when the first medical unit 14 is brought into proximity with the display monitor 12. In the same or a different implementation, the second medical unit 32 is adapted to automatically wirelessly connect to the display monitor 12 when the second medical unit 32 is brought into proximity with the display monitor 12.

In one construction of the fourth expression of the embodiment of FIGS. 1-2, the second medical unit 32 includes a second IV (intravenous) pump assembly 56 which is connected to the wireless connection module 34 of the second medical unit 32 by a wire 50, which is controllable from the user interface 46 of the display monitor 12, and which propels the second drug 54 through a second IV tube 58 to the patient 16. In one variation, the wireless connection module 34 of the second medical unit 32 sends a wireless signal or segment thereof which includes an identification portion which is recognized by the wireless connection module 28 of the display monitor 12 as a signal from a second IV pump assembly 56 and which includes a flow rate data portion recognizable as such by the wireless connection module 28 of the display monitor 12.

FIG. 2 shows one illustration of a screen display for the medical system 10 embodiment of FIG. 1. In this illustration, the screen 30 of the display monitor 12 of the medical system 10 displays: “PULSE=75” as an example of a pulse rate of 75 beats per minute; “SPO2=80” as an example of a saturation of peripheral oxygen level of 80%; “SYS BP=140” as an example of a systolic blood pressure level of 140 mmHg; “FLOW1=0.03” as an example of a flow rate of the first drug 44 of 0.03 cc/min; and “FLOW2=0.05” as an example of a flow rate of the second drug 54 of 0.05 cc/min.

Examples, without limitation, of a user interface 46 of the display monitor 12 include a touch screen, voice recognition, and a keypad. Examples of other physiological parameters of the patient 16 and modules for measuring them, as well as other types of drugs and other types of user interfaces, are left to those skilled in the art.

Several benefits and advantages are obtained from one or more of the expressions of the embodiment of the invention. In one example, the wireless connection reduces the number of cables present during a medical procedure. In the same or a different example, each medical unit is adapted to automatically wirelessly connect to the display monitor when brought into proximity with the display monitor which reduces the time the procedure room is occupied to perform a medical procedure on a patient.

While the present invention has been illustrated by several expressions of an embodiment, and enablements, implementations, etc. thereof, it is not the intention of the applicants to restrict or limit the spirit and scope of the appended claims to such detail. Numerous other variations, changes, and substitutions will occur to those skilled in the art without departing from the scope of the invention. It will be understood that the foregoing description is provided by way of example, and that other modifications may occur to those skilled in the art without departing from the scope and spirit of the appended Claims.

What is claimed is:

1. A medical system comprising:
a) a display monitor; and
b) a first medical unit which is adapted to measure a first physiological parameter of a patient and which is adapted to wirelessly connect to the display monitor, wherein the display monitor is adapted to display the first physiological parameter.

2. The medical system of claim 1, wherein the first medical unit is adapted to automatically wirelessly connect to the display monitor when the first medical unit is brought into proximity with the display monitor.

3. The medical system of claim 1, wherein the first medical unit is adapted to measure an additional physiological parameter of the patient different from the first physiological parameter, and wherein the display monitor is adapted to display the additional physiological parameter.

4. The medical system of claim 3, wherein the first physiological parameter is a pulse rate of the patient, and wherein the additional physiological parameter is an SpO₂ level of the patient.

5. The medical system of claim 4, wherein the first medical unit is adapted to automatically wirelessly connect to the display monitor when the first medical unit is brought into proximity with the display monitor.

6. A medical system comprising:
a) a display monitor;
b) a first medical unit which is adapted to measure a first physiological parameter of a patient and which is adapted to wirelessly connect to the display monitor, wherein the display monitor is adapted to display the first physiological parameter; and
c) a second medical unit which is adapted to measure a second physiological parameter different from the first physiological parameter and which is adapted to wirelessly connect to the display monitor separately from the wireless connection of the first medical unit to the display monitor, wherein the display monitor is adapted to display the second physiological parameter.

7. The medical system of claim 6, wherein the first physiological parameter is a pulse rate of the patient, and wherein the second physiological parameter is a systolic blood pressure level of the patient.

8. The medical system of claim 7, wherein the first medical unit is adapted to automatically wirelessly connect to the display monitor when the first medical unit is brought into proximity with the display monitor.

9. The medical system of claim 8, wherein the second medical unit is adapted to automatically wirelessly connect to the display monitor when the second medical unit is brought into proximity with the display monitor.

10. A medical system comprising:
a) a display monitor; and
b) a first medical unit which is adapted to measure a first physiological parameter of a patient and to deliver a first drug to the patient and which is adapted to wirelessly connect to the display monitor, wherein the display monitor is adapted to display the first physiological parameter and a flow rate of the first drug.

11. The medical system of claim 10 wherein the wireless connection of the first medical unit to the display monitor is a two-way connection, and wherein the display monitor includes a user interface for changing the flow rate of the first drug.

12. The medical system of claim 11, wherein the delivered drug is a sedation drug.

13. The medical system of claim 12, wherein the first physiological parameter is a pulse rate of the patient.

14. The medical system of claim 13, wherein the first medical unit is adapted to automatically wirelessly connect to the display monitor when the first medical unit is brought into proximity with the display monitor.

15. A medical system comprising:
a) a display monitor;
b) a first medical unit which is adapted to measure a first physiological parameter of a patient and which is adapted to wirelessly connect to the display monitor, wherein the display monitor is adapted to display the first physiological parameter; and
c) a second medical unit which is adapted to deliver a second drug to the patient and which is adapted to wirelessly connect to the display monitor separately from the wireless connection of the first medical unit to the display monitor, wherein the display monitor is adapted to display the flow rate of the second drug.

16. The medical system of claim 15, wherein the wireless connection of the second medical unit to the display monitor is a two-way connection, and wherein the display monitor includes a user interface for changing the flow rate of the second drug.

17. The medical system of claim 16, wherein the delivered drug is a sedation drug.

18. The medical system of claim 17, wherein the first physiological parameter is a pulse rate of the patient.

19. The medical system of claim 18, wherein the first medical unit is adapted to automatically wirelessly connect to the display monitor when the first medical unit is brought into proximity with the display monitor.

20. The medical system of claim 19, wherein the second medical unit is adapted to automatically wirelessly connect to the display monitor when the second medical unit is brought into proximity with the display monitor.
